(19) United States
(12) Patent Application Publication
(10) Pub. No.: US 2008/0281243 A1
(43) Pub. Date: Nov. 13, 2008

(54) ADJUSTABLE JOINT SPLINT

(76) Inventor: Chih-Hao CHANG, Taipei (TW)

Correspondence Address: PATENTTM.US, P. O. BOX 82788, PORTLAND, OR, 97282-0788, US

(21) Appl. No.: 11/746,883

(22) Filed: May 10, 2007

(51) Int. Cl. A61F 5/00

(52) U.S. Cl. 602/22

(57) ABSTRACT

An adjustable joint splint has a splint, two pads and an adjustable strap. The splint has two curved end segments and two connectors. The curved end segments are curved in the same direction and are parallel to each other, and each has an inner surface. The connectors connect the curved end segments. The pads are mounted respectively on the inner surfaces of the curved end segments. The adjustable strap is mounted between the connectors. Doctors can easily adjust the adjustable strap and the splint to conform to joints of different patients, patients can also tighten or loosen the strap around their joints themselves, and the splint has an opening that provides good ventilation. Therefore joints are held securely and comfortably by the splint, and skin in the area of the immobilized joint does not become irritated.

### BACKGROUND OF THE INVENTION

1. Field of the Invention

The present invention relates to an adjustable joint splint.

2. Description of the Prior Art

Traditionally, plaster casts and splints have been used to immobilize injured joints so fractured or broken bones and strained or torn ligaments can heal.

However, casts have a number of inherent problems. First, applying and removing a cast is a problem because a trained technician is required to apply the cast and a special saw is required to remove the cast. Second, open wounds associated with the injury cannot be treated and cleaned with a cast in place, and almost all ventilation is cut off to the injured area. Furthermore, casts tend to rub against and irritate skin inside the cast when the cast must be worn for a long time.

To overcome the problems associated with casts, a removable cast was developed and can be mounted around and immobilize a joint. Although the removable casts are easy to apply and remove from joints, the removable casts must be made in many sizes and shapes to allow doctors to treat injuries on different patients. Consequently, exact fits are seldom achieved for individual patients.

To overcome the shortcomings, the present invention provides an adjustable joint splint to mitigate or obviate the aforementioned problems.

### SUMMARY OF THE INVENTION

The main objective of the present invention is to provide an adjustable joint splint that has a splint, two pads and an adjustable strap. The splint is malleable and has two curved end segments and two connectors. The curved end segments are curved in the same direction and are parallel to each other, and each curved end segment has two edges and an inner surface. The connectors connect the two curved end segments and are formed between corresponding edges of the two curved end segments. The pads are mounted respectively on the inner surfaces of the two curved end segments. The adjustable strap is mounted around the connectors to hold a person's limb in the splint. Doctors can easily adjust the adjustable strap and the splint to conform to various joints, patients can also tighten or loosen the strap around their joints themselves, and the splint has an opening between the curved end segments and the connectors that provides good ventilation. Therefore, skin on and around joints immobilized by the adjustable joint splint will not become irritated.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an adjustable joint splint in accordance with the present invention;

FIG. 2 is an exploded perspective view of the adjustable joint splint in FIG. 1;

FIG. 3 is an operational perspective view of the adjustable joint splint in FIG. 1; and

FIG. 4 is an operational perspective view of the adjustable joint splint in FIG. 1 on a person's finger.

### DETAILED DESCRIPTION OF THE PREFERRED EMBODIMENTS

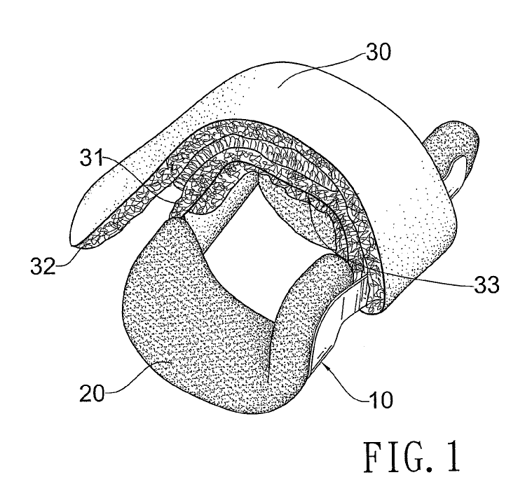

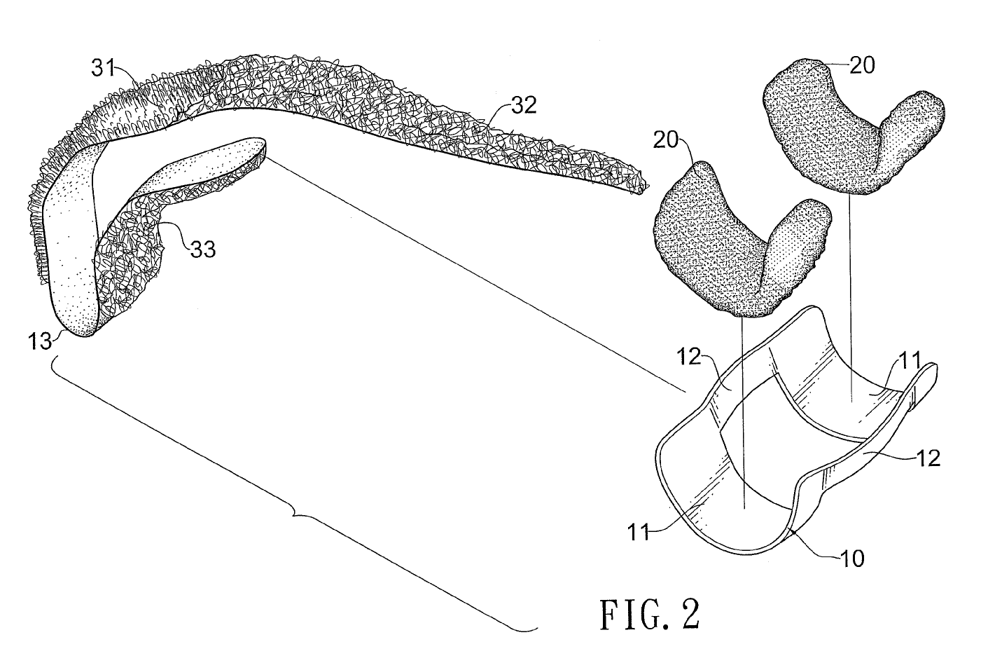

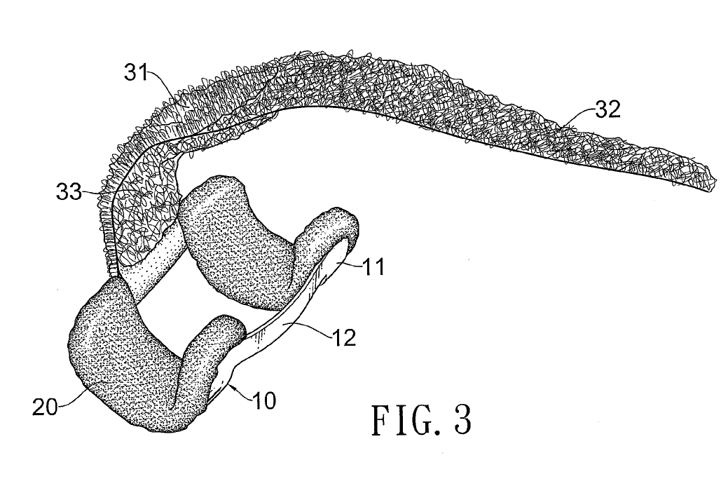

With reference to FIGS. 1 to 3, the adjustable joint splint has a splint (10), two pads (20) and an adjustable strap (30).

The splint (10) is malleable, is a somewhat semicylindrical frame, may be metal, plastic or the like and comprises two curved end segments (11) and two connectors (12).

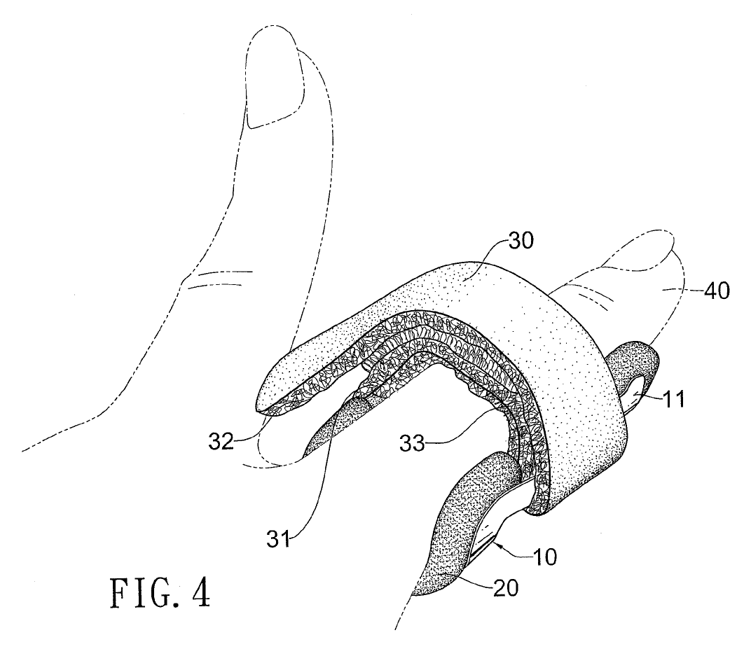

With further reference to FIG. 4, the curved end segments (11) are flat semicylindrical rings, are parallel to each other, are curved in the same direction and are mounted on opposite sides of a person's joint, and each curved end segment (11) has two edges and an inner surface.

The two connectors (12) connect the two curved end segments (11) and are formed between corresponding edges of the two curved end segments (11), and each connector (12) has two ends formed respectively on the curved end segments (11) at corresponding edges.

The two pads (20) are mounted respectively on the inner surfaces of the two curved end segments (11).

The adjustable strap (30) is connected securely to one of the connectors (12), is connected detachably to the opposite connector (12), holds a person's joint securely in the splint (10), comprises two ends, an outer surface, an inner surface, a hooked surface (31) and a looped surface (32) and may have a padded surface (33). The hooked surface (31) is formed on the outer surface of the adjustable strap (30) at the end that connects securely to one connector (12). The looped surface (32) is formed on the outer surface of the adjustable strap (30) adjacent to the hooked surface (31) and the other end, is mounted around the other connector (12) and connects adjustably to the hooked surface (31). The padded surface (33) is formed on the inner surface of the adjustable strap (30) and opposite to the hooked surface (31).

The adjustable joint splint as described has the following advantages. The splint (10) can be bent to fit fingers (40) of different people and has an opening that provides good ventilation to prevent the skin from becoming irritated. Therefore the finger (40) is comfortably mounted between the two pads (20) and the padded surface (33).

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An adjustable joint splint having:
a splint being malleable and comprising
two curved end segments being flat semicylindrical rings, being parallel to each other and being curved in a same direction, and each curved end segments having
two edges; and
an inner surface; and
two connectors connecting between the curved end segments;
two pads being mounted respectively on the inner surfaces of the two curved end segments; and
an adjustable strap being connected securely to one of connectors, being connected detachably to the other connector.

2. The adjustable joint splint as claimed in claim 1, wherein the splint is a semicylindrical frame.

3. The adjustable joint splint as claimed in claim 1, wherein the connectors are formed between the edges of the curved end segments.

4. The adjustable joint splint as claimed in claim 1, wherein the adjustable strap comprises
two ends;
an outer surface;
an inner surface;
a hooked surface being formed on the outer surface of the adjustable strap at the end that connects securely to one of the connectors; and
a looped surface being formed on the outer surface of the adjustable strap adjacent to the hooked surface and the other end of the adjustable strap, being mounted around the other connector and connecting adjustably to the hooked surface.

5. The adjustable joint splint as claimed in claim 2, wherein the adjustable strap comprises
two ends;
an outer surface;
an inner surface;
a hooked surface being formed on the outer surface of the adjustable strap at the end that connects securely to one of the connectors; and
a looped surface being formed on the outer surface of the adjustable strap adjacent to the hooked surface and the other end of the adjustable strap, being mounted around the other connector and connecting adjustably to the hooked surface.

6. The adjustable joint splint as claimed in claim 3, wherein the adjustable strap comprises
two ends;
an outer surface;
an inner surface;
a hooked surface being formed on the outer surface of the adjustable strap at the end that connects securely to one of the connectors; and
a looped surface being formed on the outer surface of the adjustable strap adjacent to the hooked surface and the other end of the adjustable strap, being mounted around the other connector and connecting adjustably to the hooked surface.

7. The adjustable joint splint as claimed in claim 4, wherein the adjustable strap further has a padded surface formed on the inner surface of the adjustable strap and opposite to the hooked surface.

8. The adjustable joint splint as claimed in claim 5, wherein the adjustable strap further has a padded surface formed on the inner surface of the adjustable strap and opposite to the hooked surface.

9. The adjustable joint splint as claimed in claim 6, wherein the adjustable strap further has a padded surface formed on the inner surface of the adjustable strap and opposite to the hooked surface.

10. The adjustable joint splint as claimed in claim 1, wherein the splint is metal.

11. The adjustable joint splint as claimed in claim 1, wherein the splint is plastic.
